(19) United States
(12) Patent Application Publication
(10) Pub. No.: US 2024/0301337 A1
(43) Pub. Date: Sep. 12, 2024

(54) BIOCHIP AND METHOD OF MANUFACTURING THE SAME

(71) Applicant: DONGGUK UNIVERSITY WISE CAMPUS INDUSTRY-ACADEMY COOPERATION FOUNDATION, Gyeongju-si (KR)

(72) Inventors: BONG SEOP KWAK, Goyang-si (KR); SUNG HAN LEE, Goyang-si (KR); JAE HUN LEE, Goyang-si (KR); YEONG WON KIM, Goyang-si (KR)

(73) Assignee: DONGGUK UNIVERSITY WISE CAMPUS INDUSTRY-ACADEMY COOPERATION FOUNDATION, Gyeongju-si (KR)

(21) Appl. No.: 18/597,848

(22) Filed: Mar. 6, 2024

(30) Foreign Application Priority Data
Mar. 9, 2023 (KR) 10-2023-0030948

(51) Int. Cl. C12M 3/06; B01L 3/00; C12M 3/00

(52) U.S. Cl. CPC C12M 23/16; B01L 3/502753; C12M 21/08; B01L 2300/0819

(57) ABSTRACT

A biochip includes a first chip body configured to culture cells or biological tissues inside a first channel formed in the first chip body and including a first contact channel portion formed on one side communicating with the channel to be in close contact with a second contact channel portion of a second chip body; and a second chip body configured to culture heterogeneous cells or biological tissues, which are different from the cells or biological tissues cultured in the first chip body, in a second channel formed inside the second chip body and including the second chip body which is formed on a side facing the first chip body and forms the second contact channel portion in close contact with the first contact channel portion of the first chip body.

### CROSS-REFERENCE TO RELATED APPLICATION

This application is based on and claims priority under 35 U.S.C. § 119 to Korean Patent Application No. 10-2023-0030948, filed on Mar. 9, 2023, in the Korean Intellectual Property Office, the disclosure of which is incorporated by reference herein in its entirety.

### BACKGROUND

The present disclosure relates to a biochip and a method of manufacturing the biochip, and particularly, to a biochip and a method of manufacturing the biochip in which one biochip body is divided into a first chip body and a second chip body based on a virtual boundary, a blood vessel simulation chip is manufactured which simulates blood vessels by using the first chip body, a tumor culture chip in which tumor cells are cultured is manufactured by using the second chip body, and the blood vessel simulation chip and the tumor culture chip manufactured respectively are brought into close contact such that the drug introduced through the blood vessel simulation chip is transferred to the tumor culture chip.

In the well-known art, it is common to culture single cell species, and co-culture or co-culture systems for growing different cells are also common for growing animal cells together.

That is, it is difficult to find a co-culture system that simultaneously cultures eukaryotic cells, such as animal cells or plant cells, and bacteria (prokaryotic cells).

In addition, when eukaryotic cells and bacteria are co-cultured, bacteria are far superior in metabolic rate and proliferation rate, and accordingly, bacteria are the dominant species in a culture system.

This means that the resources available to animal cells are rapidly lost in the metabolism of nutrients in a culture medium or medium.

Also, eukaryotic cells are likely to stop dividing or die due to direct contact with bacteria.

Recently, there has been increased in demand to explore the effects of bacterial flora on human cells, tissues, organs, and individual units through microbiome research.

However, due to the absence of such a co-culture system, microbiome research generally involves experiments using extracts or animal experiments through direct inoculation.

In the case of extract research, a dynamic change in a bacterial growth process may not be directly captured in human cells or tissues, and in bacterial flora research involving multiple bacteria, it is difficult to combine single compounds or extracts.

Because animal test has ethical issues as well as high costs, there are limitations in large-scale exploratory work such as material screening studies.

Example of the well-known art include Korean Patent Publication No. 10-2014-0076212 (Jun. 20, 2014).

### SUMMARY

The present disclosure provides a biochip and a method of manufacturing the biochip in which one biochip body is divided into a first chip body and a second chip body based on a virtual boundary, heterogeneous cells or biological tissues with different culture environments may be cultured (single culture) in separate culture environments in each of the first and second chip bodies, open regions of the first chip body and the second chip body may be bonded to each other after cells or biological tissues are respectively cultured, and drug is easily transferred between separately cultured chip bodies.

A biochip according to the present disclosure includes a first chip body configured to culture cells or biological tissues inside a first channel formed in the first chip body and including a first contact channel portion formed on one side communicating with the channel to be in close contact with a second contact channel portion of a second chip body, and a second chip body configured to culture heterogeneous cells or biological tissues, which are different from the cells or biological tissues cultured in the first chip body, in a second channel formed inside the second chip body and including the second chip body which is formed on a side facing the first chip body and forms the second contact channel portion in close contact with the first contact channel portion of the first chip body.

In this case, the first chip body according to the present disclosure includes a first inlet through which a fluid is introduced from the outside, a first guide channel having one side connected to the first inlet and configured to guide a fluid introduced through the first inlet to the other side, a first outlet connected to the other side of the first guide channel and discharging a fluid flowing along the first guide channel to the outside, and a first contact channel portion having one side communicating with the first guide channel and having the other side on which a first open channel communicating with the outside is formed.

Here, according to the present disclosure, a blood vessel simulation chip may be configured to form a basement membrane in an open region, which communicates with the outside, in the first open channel of the first contact channel portion and may simulate blood vessels by forming a vascular endothelial cell membrane on an inner surface of the base membrane.

The second chip body according to the present disclosure includes a second inlet through which a fluid is introduced from the outside, a second guide channel having one side connected to the second inlet and configured to guide a fluid introduced through the second inlet to the other side, a second outlet connected to the other side of the second guide channel and discharging a fluid flowing along the second guide channel to the outside, and a second contact channel portion having one side communicating with the second guide channel and having the other side on which a second open channel communicating with the outside is formed.

In this case, a tumor culture chip may be configured by forming an extracellular matrix including tumor cells in the second open channel of the second contact channel portion.

Also, the biochip according to the present disclosure may further include a bonding maintenance tray configured to accommodate the first chip body and the second chip body and move the first chip body and the second chip body in a direction facing each other to maintain a bonding state of the first chip body and the second chip body.

A method of manufacturing a biochip according to the present disclosure includes manufacturing a body of the biochip, cutting the body of the manufactured biochip and dividing the body into a first chip body and a second chip body, manufacturing a blood vessel simulation chip by using the first chip body, manufacturing a tumor culture chip by using the second chip body, and reconnect the first chip body and the second chip body using a bonding maintenance tray.

In this case, according to the present disclosure, the manufacturing of the blood vessel simulation chip by using the first chip body includes forming a basement membrane in an open region of a first open channel formed in the first chip body, and forming a vascular endothelial cell membrane on an inner surface of the basement membrane.

In addition, according to the present disclosure, the manufacturing of the tumor culture chip by using the second chip body includes forming a tumor model made of an extracellular matrix including tumor cells in the second guide channel and the second open channel formed in the second chip body.

### BRIEF DESCRIPTION OF THE DRAWINGS

Embodiments of the inventive concept will be more clearly understood from the following detailed description taken in conjunction with the accompanying drawings in which:

FIG. 1 is an example view illustrating a biochip according to an embodiment of the present disclosure;

FIG. 2 is an example view illustrating a state in which a biochip body is divided into a first chip body and a second chip body based on a virtual boundary, according to an embodiment of the present disclosure;

FIG. 3 is an example view illustrating a process of manufacturing a blood vessel simulation chip that is a first chip body of a biochip, according to an embodiment of the present disclosure;

FIG. 4 is an example view illustrating a process of manufacturing a tumor culture chip that is a second chip body of a biochip, according to an embodiment of the present disclosure;

FIG. 5 is an example view illustrating a state in which a first chip body of a biochip forms a blood vessel simulation chip and a second chip body of the biochip forms a tumor culture chip, according to an embodiment of the present disclosure;

FIG. 6 is an example view illustrating a state in which a first chip body of a biochip is bonded to a second chip body of the biochip, according to an embodiment of the present disclosure; and

FIG. 7 is a photograph illustrating a state in which drug is introduced into a first chip body of a biochip and transferred to a second chip body of the biochip, according to an embodiment of the present disclosure.

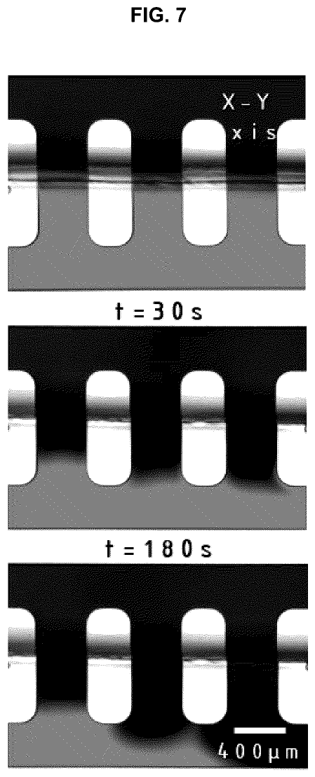

### DETAILED DESCRIPTION OF THE EMBODIMENTS

Hereinafter, preferred embodiments according to the present disclosure are described in detail with reference to the attached drawings. Prior to this, the terms or words used in the present specification and claims should not be construed as limited to their usual or dictionary meanings, and the inventor should interpret the meaning and concept consistent with the technical idea of the present disclosure based on the principle that the inventor may define terms appropriately in order to describe the present disclosure in the best possible way.

Therefore, the embodiments described in the present specification and the configurations illustrated in the drawings are only the most preferred examples of the present disclosure and do not represent the entire technical idea of the present disclosure, and accordingly, it should be understood that there may be equivalent modifications capable of replacing the example at the time of filing the present application.

The present disclosure relates to a biochip and a method of manufacturing the biochip in which one biochip body is divided into a first chip body and a second chip body based on a virtual boundary, a blood vessel simulation chip is manufactured which simulates blood vessels by using the first chip body, a tumor culture chip in which tumor cells are cultured is manufactured by using the second chip body, and the blood vessel simulation chip and the tumor culture chip manufactured respectively are brought into close contact such that the drug introduced through the blood vessel simulation chip is transferred to the tumor culture chip, and is described below in detail with reference to the drawings.

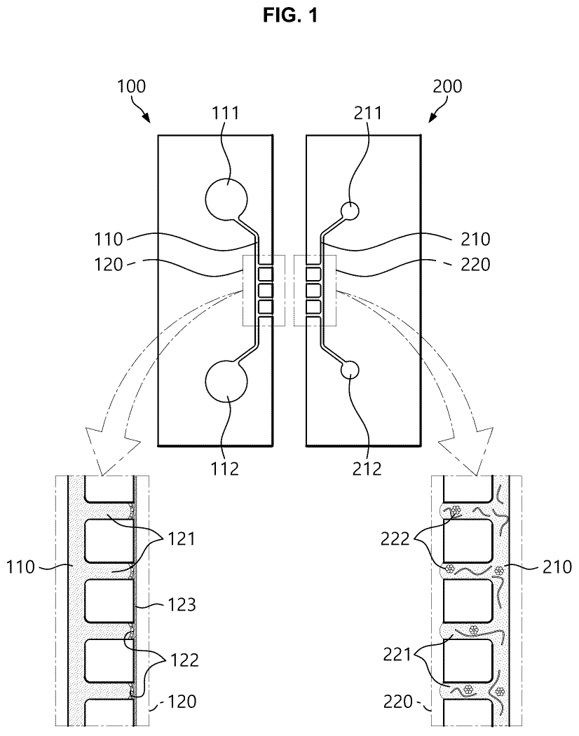

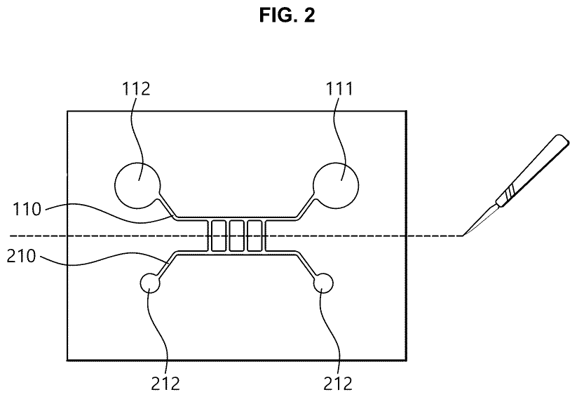

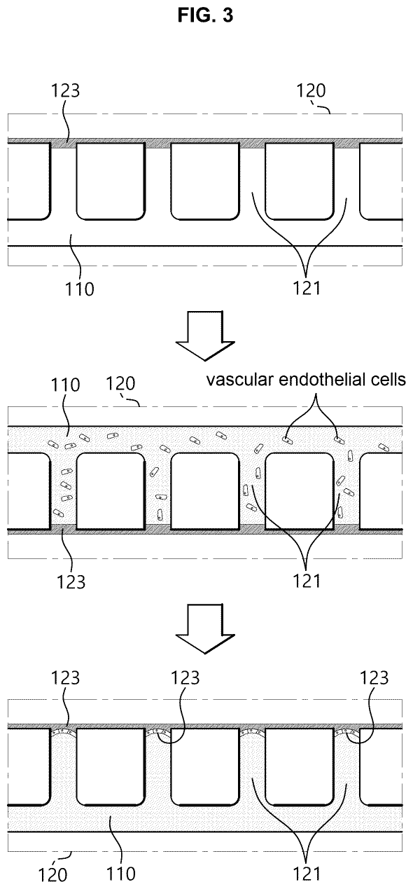

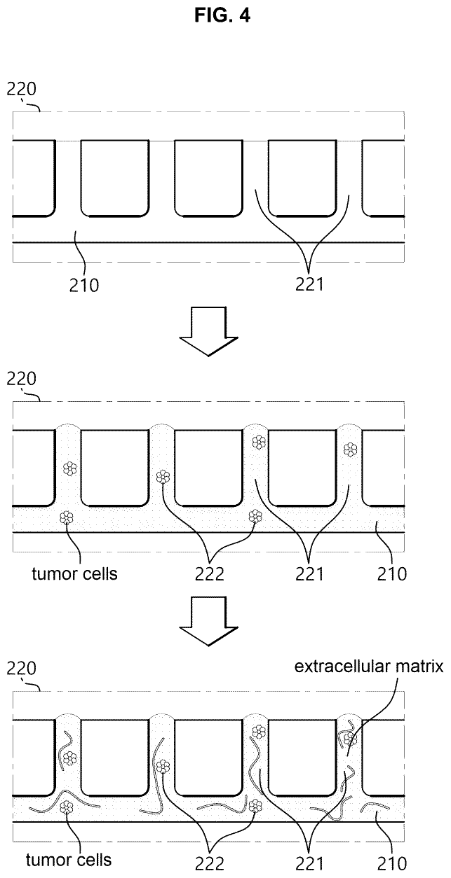

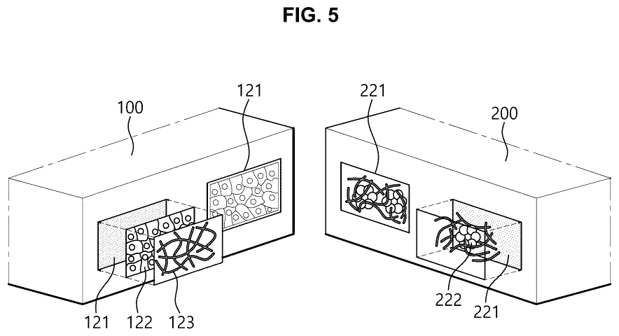

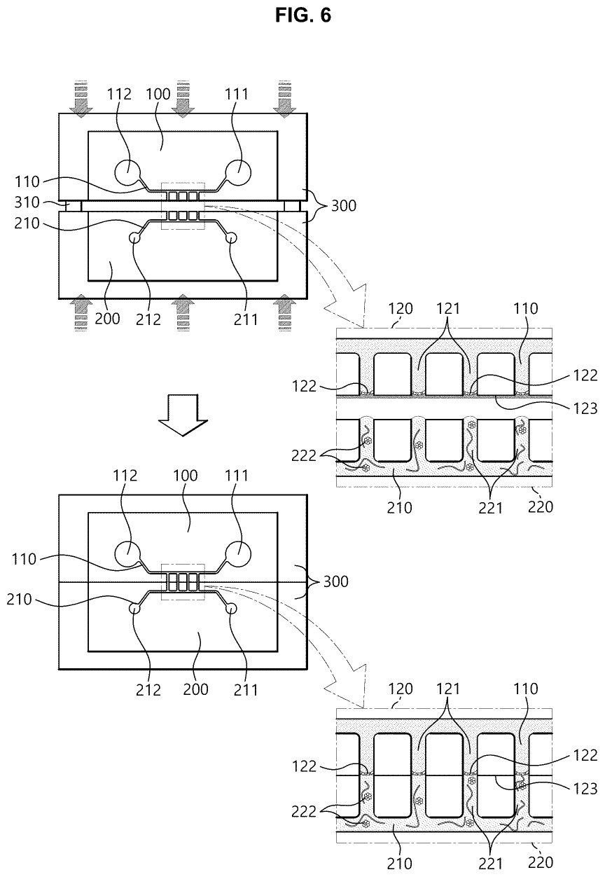

Referring to FIGS. 1 to 6, a biochip according to an embodiment of the present disclosure includes a first chip body 100 and a second chip body 200, and the first chip body 100 and the second chip body 200 one biochip body are respective bodies obtained by cutting one biochip body into two chip bodies based on a virtual boundary.

The first chip body 100 includes cells or biological tissues cultured in a first channel formed inside, and a first contact channel portion 120, which is close to a second contact channel portion 220 of the second chip body 200, is formed on one side communicating with the channel.

The second chip body 200 includes heterogeneous cells or biological tissues which are cultured in a second channel formed inside and are different from the cells or biological tissues cultured in the first chip body 100, and the second contact channel portion 220, which is close to the first contact channel portion 120 of the first chip body 100, is formed on a side facing the first chip body 100.

The first chip body 100 and the second chip body 200 are described below in more detail, and first, the first chip body 100 has an overall shape of a hexahedron and includes a first guide channel 110 that guides fluids, such as a culture medium and blood, to flow horizontally from one side to the other side.

A first inlet 111 through which a fluid is introduced from the outside is formed on one side of the first guide channel 110, and a first outlet 112 is formed on the other side of the first guide channel 110 such that a fluid flowing along the first guide channel 110 may be discharged to the outside.

Therefore, the first inlet 111 and the first outlet 112 are each formed on one of upper surfaces of the first chip body 100, and when a fluid is introduced from the outside through the first inlet 111, the fluid introduced through the first inlet 111 flows from one side to the other side along the first guide channel 110 and is discharged to the outside through the first outlet 112.

In addition, a first contact channel portion 120 in contact with the second contact channel portion 220 of the second chip body 200 is formed in a portion in contact with the second chip body 200 among side surfaces of the first chip body 100.

In this case, a plurality of first open channels 121 communicating with the first guide channel 110 are formed in the first contact channel portion 120, and each of the plurality of first open channels 121 preferably has a longitudinal direction that is perpendicular to a longitudinal direction of the first guide channel 110.

Here, the first chip body 100 has an open region, which is at the end of the first open channel 121 and communicates with the outside and in which a vascular endothelial cell membrane 122 is formed, to implement a blood vessel simulation chip, and accordingly, leakage of fluid (blood) flowing along the first guide channel 110 and the first open channel 121 is prevented, and vascular endothelium is simulated.

In this case, the vascular endothelial cell membrane 122 is formed to be supported by a basement membrane 123 provided in the open region of the first open channel 121.

Therefore, the basement membrane 123 is formed in the open region of the first open channel 121 by applying collagen for forming the basement membrane 123 to the open regions of the first open channels 121 and curing the collagen, and then a culture medium including vascular endothelial cells is introduced through the first inlet 111 and flows along the first guide channel 110.

In addition, in order for the vascular endothelial cells to be attached to the basement membrane 123 formed in the open regions of the first open channels 121, a width of the first chip body 100 is erected in a vertical direction such that the vascular endothelial cells are collected in the first open channels 121.

Thereafter, the vascular endothelial cells are cultured again with the width of the first chip body 100 facing a horizontal direction such that the vascular endothelial cell membrane 122 is formed in the open regions of the first open channels 121, and accordingly, the first chip body 100 is manufactured as a blood vessel simulation chip.

In addition, the second chip body 200 also has a hexahedral shape, and a second guide channel 210 is formed inside the second chip body 200 to guide a fluid, such as a culture medium, to flow in the horizontal direction from one side to the other side.

A second inlet 211, through which a fluid is introduced from the outside, is formed on one side of the second guide channel 210, and a second outlet 212 is formed on the other side of the second guide channel 210 such that a fluid flowing along the second guide channel 210 may be discharged to the outside.

Therefore, the second inlet 211 and the second outlet 212 are each formed on one side of upper surfaces of the second chip body 200, and when a fluid is introduced from the outside through the second inlet 211, the fluid introduced through the second inlet 211 flows from one side to the other side along the second guide channel 210 and is discharged to the outside through the second outlet 212.

In addition, the second contact channel portion 220 in contact with the first contact channel portion 120 of the first chip body 100 is formed in a portion in contact with the first chip body 100 among side surfaces of the second chip body 200.

In this case, a plurality of second open channels 221 communicating with the second guide channel 210 are formed in the second contact channel portion 220, and each of the plurality of second open channels 221 preferably has a longitudinal direction that is perpendicular to a longitudinal direction of the second guide channel 210.

Here, the second chip body 200 includes a tumor model 222 inside the second guide channel 210 and the second open channel 221 to implement a tumor culture chip.

The tumor model 222 includes a large number of tumor cells in an extracellular matrix (ECM), and after a collagen solution mixed with the tumor cells is introduced into the second guide channel 210 and the second open channel 221 through the second inlet 211, the introduced collagen solution is cured at 37° C. for a certain period of time, the solution is changed into polymer and fiber to form an extracellular matrix, and accordingly, the tumor model 222 is formed.

Among the tumor models 222, tumor cells do not flow in the second open channel 221 by the extracellular matrix.

Here, positions of the tumor cells placed inside the second open channel 221 may be adjusted by a device inserted through an open region of the second open channel 221.

In addition, a sample in which a drug reaction is completed may be easily collected through the second open channel 221 of the second contact channel portion 220 by using a device, and for example, when a reaction to an anticancer drug is completed, the first chip body 100 and the second chip body 200 are separated again, and a sample of the tumor model 222 may be retrieved through the second open channel 221 of the second contact channel portion 220 (sample retrieving).

In this case, the retrieved sample may later be verified to check an effective effect of the drug.

The biochip according to an embodiment of the present disclosure may check a process, in which the drug introduced into the first chip body 100 that is a blood vessel simulation chip is transferred to the second chip body 200 that is a tumor culture chip, by using the first chip body 100 forming the blood vessel simulation chip and the second chip body 200 forming the tumor culture chip.

In this case, the first contact channel portion 120 of the first chip body 100 that is a blood vessel simulation chip is placed to face the second contact channel portion 220 of the second chip body 200 that is a tumor culture chip, and then the first chip body 100 that is the blood vessel simulation chip comes into contact with the second chip body 200 that is the tumor culture chip such that the first contact channel portion 120 of the first chip body 100 that is the blood vessel simulation chip is bonded to the second contact channel portion 220 of the second chip body 200 that is the tumor culture chip.

Here, a bonding maintenance tray 300 is used to maintain a bonding state of the first chip body 100 that is a blood vessel simulation chip and the second chip body 200 that is a tumor culture chip, the bonding maintenance tray 300 has a shape of a square plate and is divided into a left bonding maintenance tray 300 and a right bonding maintenance tray 300, accommodation spaces for accommodate the blood vessel simulation chip 100 and the tumor culture chip 200 are formed in upper surfaces of the left bonding maintenance tray 300 and the right bonding maintenance tray 300, and the left bonding maintenance tray 300 and the right bonding maintenance tray 300 move in the horizontal direction according to the guidance of a tray guide 310 extending laterally from any one of the left bonding maintenance tray 300 and the right bonding maintenance tray 300.

Therefore, the first chip body 100 that a blood vessel simulation chip and the second chip body 200 that is a tumor culture chip are accommodated in the accommodation space of each of the left bonding maintenance tray 300 and the right bonding maintenance tray 300, and as the first chip body 100 and the second chip body 200 move horizontally, the first chip body 100 that is a blood vessel simulation chip and the second chip body 200 that is a tumor culture chip come into close contact with each other to be bonded together and may maintain the bonding state.

A method of manufacturing a biochip according to an embodiment of the present disclosure includes a) a step of manufacturing a body of the biochip, b) a step of cutting the body of the manufactured biochip and dividing into a first chip body and a second chip body, c) a step of manufacturing a blood vessel simulation chip by using the first chip body of the bisected bodies, and d) a step of manufacturing a tumor culture chip by using the second chip body of the bisected bodies.

In more detail, first, the body of the biochip is manufactured in step a).

In this case, the body of the biochip is made of a transparent polymer and may be manufactured by three-dimensionally printing polymer to a shape corresponding to the body of the biochip or may be manufactured by introducing liquid polydimethylsiloxane (PDMS) into a mold of a shape corresponding to the body of the biochip and curing the liquid PDMS.

Here, when the body of the biochip is manufactured, it is preferable that the first chip body 100 that is a blood vessel simulation chip and the second chip body 200 that is a tumor culture chip are integrally formed to form one body, and it is preferable that the first guide channel 110 of the first chip body 100, the first inlet 111, the first outlet 112, the first open channel 121 of the first contact channel portion 120, the second guide channel 210 of the second chip body 200, the second inlet 211, the second outlet 212, and the second open channel 221 of the second contact channel portion 220 are formed together in the body.

Therefore, when the body of the biochip is manufactured, the first open channel 121 of the first contact channel portion 120 in the first chip body 100 that is a blood vessel simulation chip preferably communicates with the second open channel 221 of the second contact channel portion 220 in the second chip body 200 that is a tumor culture chip, and the first chip body 100 that is a blood vessel simulation chip is preferably symmetrical to the second chip body that is a tumor culture chip 200 based on a boundary (center line) between the first contact channel portion 120 and the second contact channel portion 220.

Next, in step b), the manufactured biochip is cut by using the center thereof as a boundary to be divided into the first chip body 100 and the second chip body 200.

In this case, a virtual center line is selected based on the center of the first contact channel portion 120 of the first chip body 100 that is a blood vessel simulation chip and the second contact channel portion 220 of the second chip body 200 that is a tumor culture chip, which are symmetrical to each other, and then the biochip is divided into the first chip body 100 and the second chip body 200 by cutting along the selected center line as a boundary.

Next, in step c), a blood vessel simulation chip is manufactured by using the first chip body 100 among the bisected bodies.

In this case, the step of manufacturing the blood vessel simulation chip 100 by using the first chip body 100 includes a step of forming the basement membrane 123 and a step of forming the vascular endothelial cell membrane 122, and in the step of forming the basement membrane 123, the basement membrane 123 is formed in an open region of the first open channel 121 by applying collagen for forming the basement membrane 123 to the open region (a portion communicating with the outside) of the first open channel 121 of the first chip body 100 and curing the collagen.

In the step of forming the vascular endothelial cell membrane 122, a culture medium including vascular endothelial cells is introduced into the first inlet 111 to flow along the first guide channel 110, and a chip body width of the blood vessel simulation chip 100 is vertically erected to collect the vascular endothelial cells in the first open channels 121 such that the vascular endothelial cells are attached to the basement membrane 123 formed in the open regions of the first open channels 121.

Thereafter, the vascular endothelial cells are cultured again by causing the chip body width of the blood vessel simulation chip 100 to face a horizontal direction, and accordingly, the vascular endothelial cell membrane 122 is formed in the open regions of the first open channels 121.

Next, in step d), a tumor culture chip is manufactured by using the second chip body 200 among the bisected bodies.

In this case, in the step of manufacturing the tumor culture chip by using the second chip body 200, the tumor model 222 is formed inside the second guide channel 210 and the second open channel 221, the tumor model 222 includes a large number of tumor cells in an extracellular matrix (ECM), a collagen solution mixed with the tumor cells introduced into the second guide channel 210 and the second open channel 221 through the second inlet 211 is cured at 37° C. for a certain period of time, the collagen solution is changed into polymer and fiber to form an extracellular matrix, and the tumor cells are accommodated in the second open channel 221 by the extracellular matrix.

Therefore, in a biochip according to an embodiment of the present disclosure, one biochip body is divided into the first chip body 100 and the second chip body 200 based on a virtual boundary, heterogeneous cells or biological tissues with different culture environments may be cultured (single culture) in separate culture environments in each of the bisected first chip body 100 and the second chip body 200, cells or biological tissues are separately cultured and then bonded together, and thus, the introduced drug is easily transferred.

The well-known biochip has to be retrieved by cutting a channel, and accordingly, there is a problem that a tumor is destroyed by pressure when cutting the channel, but the first chip body 100 and the second chip body 200 respectively include the contact channel portions 120 and 220 in which open channels 121 and 221 communicating with the outside are respectively formed, and the introduced drug is easily transferred through the open channels 121 and 221.

Also, blood vessels and tumors may be fluorescently stained separately, and different biomarkers may be seen at one wavelength (photos may be taken individually or combined together).

Also, a culture environment cross-section (a width direction and number) of the first chip body 100 and the second chip body 200 may be easily viewed with a fluorescence microscope through the separated contact channel portions 120 and 220.

Effects achieved by the biochip and the method of manufacturing the biochip according to the present disclosure are as follows.

One biochip body is divided into a first chip body and a second chip body based on a virtual boundary, and heterogeneous cells or biological tissues with different culture environments may be cultured (single culture) separate environments in each of the first and second chip bodies.

Also, after cells or biological tissues are cultured separately, open regions of the first chip body and the second chip body may be bonded to each other, and thus, drug may be easily transferred between the separately cultured chip bodies.

Also, heterogeneous cells or biological tissues which are separately cultured may be fluorescently stained separately, and different biomarkers may be seen at one wavelength.

The present disclosure is described with reference to the embodiments illustrated in the drawings, but these are merely illustrative, and those skilled in the art will understand that various modifications and equivalent other embodiments may be derived therefrom. Therefore, the true scope of technical protection of the present disclosure should be determined by the technical idea of the attached patent claims.

1. A biochip comprising:
a first chip body configured to culture cells or biological tissues inside a first channel formed in the first chip body and including a first contact channel portion formed on one side communicating with the channel to be in close contact with a second contact channel portion of a second chip body; and
a second chip body configured to culture heterogeneous cells or biological tissues, which are different from the cells or biological tissues cultured in the first chip body, in a second channel formed inside the second chip body and including the second chip body which is formed on a side facing the first chip body and forms the second contact channel portion in close contact with the first contact channel portion of the first chip body.

2. The biochip of claim 1, wherein the first chip body includes:
a first inlet through which a fluid is introduced from the outside;
a first guide channel having one side connected to the first inlet and configured to guide a fluid introduced through the first inlet to the other side;
a first outlet connected to the other side of the first guide channel and discharging a fluid flowing along the first guide channel to the outside; and
a first contact channel portion having one side communicating with the first guide channel and having the other side on which a first open channel communicating with the outside is formed.

3. The biochip of claim 2, further comprising:
a blood vessel simulation chip including a basement membrane formed in an open region, which communicates with the outside, in the first open channel of the first contact channel portion and configured to simulate blood vessels by forming a vascular endothelial cell membrane on an inner surface of the base membrane.

4. The biochip of claim 1, wherein the second chip body includes:
a second inlet through which a fluid is introduced from the outside;
a second guide channel having one side connected to the second inlet and configured to guide a fluid introduced through the second inlet to the other side;
a second outlet connected to the other side of the second guide channel and discharging a fluid flowing along the second guide channel to the outside; and
a second contact channel portion having one side communicating with the second guide channel and having the other side on which a second open channel communicating with the outside is formed.

5. The biochip of claim 4, further comprising:
a tumor culture chip by forming an extracellular matrix including tumor cells in the second open channel of the second contact channel portion.

6. The biochip of claim 5, wherein
a sample, in which a drug reaction is completed, is retrieved (sample retrieving) through the second open channel of the second contact channel portion.

7. The biochip of claim 1, further comprising:
a bonding maintenance tray configured to accommodate the first chip body and the second chip body and move the first chip body and the second chip body in a direction facing each other to maintain a bonding state of the first chip body and the second chip body.

8. A method of manufacturing a biochip, the method comprising:
manufacturing a body of the biochip;
cutting the body of the manufactured biochip and dividing the body into a first chip body and a second chip body;
manufacturing a blood vessel simulation chip by using the first chip body; and
manufacturing a tumor culture chip by using the second chip body.

9. The method of claim 8, wherein the manufacturing of the blood vessel simulation chip by using the first chip body includes:
forming a basement membrane in an open region of a first open channel formed in the first chip body; and
forming a vascular endothelial cell membrane on an inner surface of the basement membrane.

10. The method of claim 8, wherein
the manufacturing of the tumor culture chip by using the second chip body includes forming a tumor model made of an extracellular matrix including tumor cells in the second guide channel and the second open channel formed in the second chip body.
